(19) United States
(12) Patent Application Publication
(10) Pub. No.: US 2011/0166234 A1
(43) Pub. Date: Jul. 7, 2011

(54) MEDICINAL PRODUCT AND TREATMENT

(76) Inventor: Hartley Campbell Atkinson, Auckland (NZ)

(21) Appl. No.: 13/062,985

(22) PCT Filed: Oct. 12, 2009
(86) PCT No.: PCT/NZ09/00220

§ 371 Date: Mar. 10, 2011

(30) Foreign Application Priority Data
Oct. 14, 2008 (NZ) 569612

(51) Int. Cl. A61K 31/192; A61P 19/02

(52) U.S. Cl. 514/570

(57) ABSTRACT

The use of paracetamol and ibuprofen in the preparation of a medicament for treating osteoarthritis or rheumatoid arthritis, wherein the medicament comprises a combination composition having approximately 125 mg to approximately 150 mg ibuprofen and approximately 475 mg to approximately 500 mg paracetamol.

### TECHNICAL FIELD

The invention relates to the use of paracetamol and ibuprofen for the treatment of osteoarthritis or rheumatoid arthritis.

### BACKGROUND

Tablets having a combination of paracetamol (about 475 mg to about 500 mg) and ibuprofen (about 125 mg to about 150 mg) are known from published patent specification WO 2006/004449 by AFT Pharmaceuticals. That WO specification discloses the use of such combination for reducing pain after dental surgery. It has now been discovered that combinations of paracetamol and ibuprofen are sufficient to give surprising synergistic results when used for reducing discomfort associated with osteoarthritis and rheumatoid arthritis.

Osteoarthritis may involve inflammation of joints resulting from abnormal wear of the cartilage which serves to cushion a joint, coupled with a decrease in the level of the synovial fluid which lubricates the joint. As bone surfaces at the joint become progressively less protected the patient suffers pain when the affected joints are made to bear normal body weight, for example when standing or walking. This can develop to the stage where muscles suffer atrophy and ligaments become lax. Rheumatoid arthritis is an autoimmune disorder which can involve the immune system attacking one's joints, also causing inflammation. It can be a disabling and painful condition leading to loss of mobility due to pain.

Paracetamol at 4,000 mg/day, taken in four doses of 1,000 mg each, is considered sufficient for low level pain or analgesic relief but for many patients it is less than effective for producing the sort of relief required for moderate to severe osteoarthritis or moderate to severe rheumatoid arthritis. It is known to treat moderate to severe osteoarthritis with ibuprofen at up to 2,400 mg per day (ie in three doses of 800 mg each). Intake of ibuprofen at that level is considered to be anti-inflammatory treatment rather than merely analgesic treatment (for analgesic treatment with ibuprofen a patient would normally only take up to 1,200 mg/day in three doses of 400 mg each). However, anti-inflammatory treatment with ibuprofen at 2,400 mg/day from three doses can result in undesirable side affects, for example adverse cardio renal conditions, thrombotic risks and gastrointestinal bleeding. Reducing the daily dose of ibuprofen reduces the risk of such side affects but at the same time gives substantially less pain and/or anti-inflammatory relief.

For many patients suffering from osteoarthritis or rheumatoid arthritis, combined ibuprofen and paracetamol treatment significantly reduces the daily dose of ibuprofen which would otherwise be necessary for achieving suitable relief. Thus the risk of side affects normally attributable to high levels of ibuprofen can be substantially reduced for some patients without at the same time compromising patient comfort, at least to any significant extent.

### SUMMARY OF THE INVENTION

According to one aspect of the invention there is provided a use of paracetamol and ibuprofen in the preparation of a medicament for treating osteoarthritis or rheumatoid arthritis, wherein the medicament comprises a combination composition having approximately 125 mg to approximately 150 mg ibuprofen and approximately 475 mg to approximately 500 mg paracetamol.

Preferably the medicament is for treating osteoarthritis.

Preferably, the composition comprises approximately 150 mg ibuprofen and approximately 500 mg paracetamol.

Preferably, the composition comprises 150 mg ibuprofen and 500 mg paracetamol. Preferably the medicament is to be taken in two dosage units up to four times each day. Preferably the medicament is to be taken in two dosage units four times each day. Preferably, the unit doses are tablets or capsules.

Preferably the medicament is presented as a pharmaceutical pack having tablets or capsules, the pack including instructions to a user to take two tablets or capsules no more than 4 times each 24 hours or at no more than 6 hourly intervals.

According to a further aspect of the invention there is provided a use of paracetamol and ibuprofen in the preparation of a medicament for treating osteoarthritis or rheumatoid arthritis, wherein the medicament comprises a combination composition comprising ibuprofen and paracetamol for administration in doses suitable to deliver approximately 250 mg to approximately 300 mg ibuprofen and approximately 950 mg to approximately 1,000 mg paracetamol per dose.

Preferably the composition is for delivering approximately 300 mg ibuprofen and approximately 1,000 mg paracetamol per dose.

Preferably the composition is for delivering 300 mg ibuprofen and 1,000 mg paracetamol per dose.

Preferably the composition is provided with instructions to the effect that 300 mg ibuprofen and 1,000 mg paracetamol be taken at each dose up four times each day.

Optionally the composition is in the form of one or more solid dosage units.

Optionally the composition is in liquid form.

According to a further aspect of the invention there is provided a method of treating osteoarthritis or rheumatoid arthritis comprising providing to a patient for consumption, or ingesting, approximately 250 mg to approximately 300 mg ibuprofen and approximately 975 mg to approximately 1,000 mg paracetamol in a single administration.

Preferably the amount of ibuprofen is approximately 300 mg and the amount of paracetamol is approximately 1,000 mg per administration.

Preferably the amount of ibuprofen is 300 mg and the amount of paracetamol is 1,000 mg per administration.

Preferably the ibuprofen and paracetamol are in the form of one or more combination tablets or capsules.

Preferably the ibuprofen and paracetamol is administered or ingested in the same way at approximately 6 hourly intervals.

According to a further aspect of the invention there is provided a use of paracetamol and ibuprofen in the preparation of a medicament for treating osteoarthritis or rheumatoid arthritis, wherein the medicament comprises a combination composition comprising a therapeutically effective amount of ibuprofen and paracetamol.

According to a further aspect of the invention there is provided a method of treating osteoarthritis or rheumatoid arthritis comprising administering, or receiving, a therapeutically effective amount of paracetamol and ibuprofen substantially simultaneously.

### BRIEF DESCRIPTION OF DRAWINGS

Some preferred embodiments of the invention will now be described by way of example and with reference to the accompanying drawings, of which:

FIG. 1 illustrates the efficacy of a preferred form of the present invention in relation to WOMAC pain scores; and

FIG. 2 further illustrates the efficacy of a preferred form of the invention in relation to Global Pain Rating scores.

### DETAILED DESCRIPTION

In a preferred form of the invention there is a tablet which has approximately 125 mg to approximately 150 mg ibuprofen and approximately 475 mg to approximately 500 mg paracetamol in combination. Most preferably the ibuprofen and paracetamol content of the tablet is approximately 150 mg and approximately 500 mg respectively. By taking two tablets every 6 hours a patient can receive a total of 1,200 mg ibuprofen and 4,000 mg paracetamol over a 24 hour period. Alternatively the tablet can be double strength so that only one tablet is required to deliver 250 mg to approximately 300 mg ibuprofen and approximately 950 mg to approximately 1,000 mg paracetamol in combination.

The level of relief obtained from two tablets taken up to four times each day is adequate for at least some patients suffering from osteoarthritis or rheumatoid arthritis, particularly the moderate to severe forms of those conditions. This is surprising because moderate to severe osteoarthritis and rheumatoid arthritis are inflammatory conditions and the level of ibuprofen and paracetamol delivered by two tablets at each dose would not be expected to significantly alleviate the discomfort caused by such inflammation. However when ibuprofen and paracetamol are combined at levels of approximately 250-300 mg and 950-1,000 mg respectively (eg from one or two tablets) a significant benefit is achieved, particularly if such treatment is repeated at 6 hourly intervals. With at least osteoarthritis there is an expectation that significantly greater levels of ibuprofen are required, for example 800 mg taken three times over a 24 hour period (ie 2,400 mg/day) for the treatment of at least moderate to severe cases. The combination therapy described herein is sufficient to deliver relief to at least some patients suffering from mild, moderate or severe osteoarthritis, and is particularly helpful to some patients in cases of moderate to severe osteoarthritis.

### Clinical Study

To exemplify the efficacy of a preferred embodiment of the invention a prospective, randomised, double-blind study was run to measure its effect on human patients suffering from osteoarthritis. Four groups of patients were selected and each group was given one or other of the following medications over a four week period:

| | | Total amount |
|---|---|---|
| | | of medication |
| Group name | Medication taken | ingested per day |
| Paracetamol | 2 × 500 mg paracetamol tablets | 4,000 mg |
| | four times daily | |
| Ibuprofen low | 2 × 150 mg ibuprofen tablets | 1,200 mg |
| | four times daily | |
| Ibuprofen high | 2 × 300 mg ibuprofen tablets | 2,400 mg |
| | four times daily | |
| combination | 2 × combination tablets four | 4,000 mg |
| | times daily, each tablet having | paracetamol |
| | 500 mg paracetamol plus 150 mg | plus 1,200 mg |
| | ibuprofen | ibuprofen |

All patients were aged from 45-80 and had been suffering from chronic knee pain due to osteoarthritis for at least 6 months. Patients went through a washout period of their existing osteoarthritis treatments. The first three groups each had 8 patients and the fourth group started with 9 patients. One of the patients in the fourth group did not complete the study. At the beginning of the study, and also at conclusion of each subsequent week, patients were required to visit a physician and complete a WOMAC questionnaire and a Global Pain Rating Assessment.

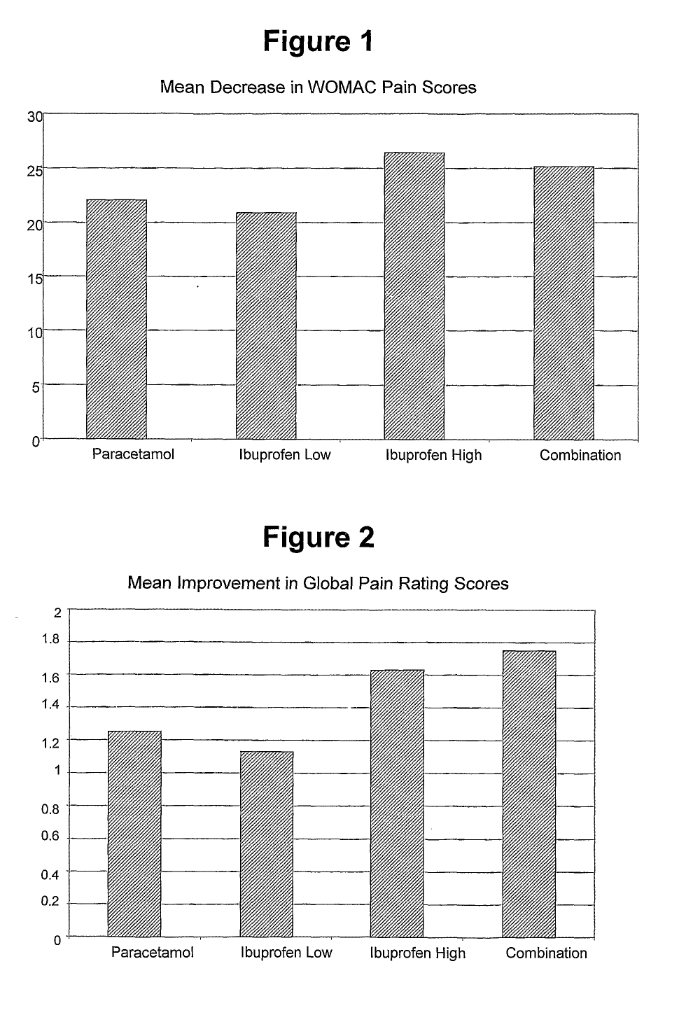

The WOMAC questionnaire involved a series of questions related to the level of pain suffered by the patients. Patients were required to mark their answers to each question separately, in a quantitative manner, using a 100 mm long analogue scale. The difference in scores between the start and end of the study was compared. The mean decrease in WOMAC pain scores is shown graphically in FIG. 1. It can be seen there that patients in the Combination group achieved significantly better pain relief than those in the Paracetamol and Ibuprofen Low groups. Indeed patients in the Combination group received virtually equivalent relief to those in the Ibuprofen High group, but without the same risks of undesirable side effects.

The Global Pain Rating Assessment involved four rating categories, namely:
Nil (no pain noticed);
Mild (pain noticed but no disruption to normal daily activity);
Moderate (pain noticed sufficient to reduce or affect daily activity);
Severe (inability to work or perform daily activity).

Patients rated their level of pain according to one or other of these categories at the start of the study, and also at the end of each subsequent week. The improvement in patient scores was assessed. For example patients who began with a “moderate” pain rating, and then at the end of the study had only a “mild” pain rating, improved by 1 rating group. By way of further example, patients who began with a severe pain rating, and then ended the study with a “mild” pain rating, improved by 2 rating groups. The mean improvement in patients in each group in terms of the number of rating groups was assessed and graphed as shown in FIG. 2. The graph shows that patients in the Combination group received significantly superior pain relief to those in the Paracetamol and Ibuprofen Low groups. It also shows that patients in the Combination group received better pain relief to those in Ibuprofen High group but, again, without the same risk of adverse side affects.

In terms of adverse side effects patients were asked to keep a record of these, if any. There were no confirmed or probable adverse side effects recorded for the Combination group, for the Paracetamol group or for Ibuprofen Low group. However in the Ibuprofen High group two probable adverse effects were recorded, both related to gastric discomfort.

It is surprising that the graphs of FIGS. 1 and 2 show the Combination providing substantially equivalent, or better, pain relief when compared to Ibuprofen High, particularly given that the Combination only involved half the amount of Ibuprofen. It is also surprising that the graphs show the Combination providing significantly better pain relief than Ibuprofen Low because both medications involved the same amount of ibuprofen and because the paracetamol in the Combination would not be expected to contribute relief to patients already taking 1,200 mg per day ibuprofen.

While effective relief for discomfort from osteoarthritis may be achieved within the first dose interval (2 tablets as described above), it is preferred to continue this with a quarterly administration regime. Two tablets or capsules four times a day is a relatively easy regime to be met by a user. Increasing from this amount may result in dosage and administration problems. This is an additional advantage over and above the potential for reduction in adverse side effects.

The tablets or capsules referred to above can be prepared and presented in the same way described in published patent specification WO 2006/004449 by AFT Pharmaceuticals, the contents of which have been incorporated herein by reference.

### Alternative Chemical Forms

While ibuprofen and paracetamol have been specifically referred to in this specification, suitable other pharmaceutically acceptable forms of the two actives (eg salts, etc) may also be used and are intended to be embraced by references to the actives per se, with the weight amounts adjusted accordingly. For example, when a salt form is used in the formulation sufficient quantity will need to be included to meet the desired amount of acid (e.g., 342 mg ibuprofen lysinate corresponds with 200 mg ibuprofen). Thus, for example, a reference to 150 mg ibuprofen may be construed as a reference to the therapeutically equivalent amount of ibuprofen lysinate.

While some preferred embodiments of the invention have been described by way of example it should be appreciated that modifications and improvements can occur without departing from the scope of the appended claims.

1. Use of paracetamol and ibuprofen in the preparation of a medicament for treating moderate or severe osteoarthritis or moderate or severe rheumatoid arthritis, wherein the medicament comprises a combination composition having approximately 125 mg to approximately 150 mg ibuprofen and approximately 475 mg to approximately 500 mg paracetamol.

2. Use according to claim 1 wherein the medicament is for treating moderate or severe osteoarthritis.

3. Use according to claim 1, wherein the medicament is for treating moderate or severe rheumatoid arthritis.

4. Use according to claim 1, wherein the composition comprises approximately 150 mg ibuprofen and approximately 500 mg paracetamol.

5. Use according to claim 1, wherein the composition comprises 150 mg ibuprofen and 500 mg paracetamol.

6. Use according to claim 1, wherein the medicament is for taking in two dosage units up to four times each day.

7. Use according to claim 1, wherein the medicament is for taking in two dosage units four times each day.

8. Use according to claim 1, wherein the medicament is for taking in two dosage units four times each day and wherein the unit doses are tablets or capsules.

9. Use according to claim 1, wherein the medicament is presented as a pharmaceutical pack having tablets or capsules, the pack including instructions to a user to take two tablets or capsules no more than 4 times each 24 hours or at no more than 6 hourly intervals.

10. Use of paracetamol and ibuprofen in the preparation of a medicament for treating moderate or severe osteoarthritis or moderate or severe rheumatoid arthritis, wherein the medicament comprises a combination composition comprising ibuprofen and paracetamol for administration in doses suitable to deliver approximately 250 mg to approximately 300 mg ibuprofen and approximately 950 mg to approximately 1,000 mg paracetamol per dose.

11. Use according to claim 10, wherein the medicament is for treating moderate or severe osteoarthritis.

12. Use according to claim 10, wherein the medicament is for treating moderate or severe rheumatoid arthritis.

13. Use according to claim 10, wherein the composition is for delivering approximately 300 mg ibuprofen and approximately 1,000 mg paracetamol per dose.

14. Use according to claim 10, wherein the composition is for delivering 300 mg ibuprofen and 1,000 mg paracetamol per dose.

15. Use according to claim 10, wherein the composition is provided with instructions to the effect that 300 mg ibuprofen and 1,000 mg paracetamol be taken at each dose up four times each day.

16. Use according to claim 10, wherein the composition is in the form of one or more solid dosage units.

17. Use according to claim 10, wherein the composition is in liquid form.

18. A method of treating moderate or severe osteoarthritis or moderate or severe rheumatoid arthritis comprising providing to a patient for consumption, or ingesting, approximately 250 mg to approximately 300 mg ibuprofen and approximately 975 mg to approximately 1,000 mg paracetamol in a single administration.

19. A method according to claim 18, wherein the method is for treating moderate or severe osteoarthritis.

20. A method according to claim 18 wherein the medicament is for treating moderate or severe rheumatoid arthritis.

21. A method according to claim 18, wherein the amount of ibuprofen is approximately 300 mg and the amount of paracetamol is approximately 1,000 mg per administration.

22. A method according to claim 18, wherein the amount of ibuprofen is 300 mg and the amount of paracetamol is 1,000 mg per administration.

23. A method according to claim 18, wherein the ibuprofen and paracetamol are in the form of one or more combination tablets or capsules.

24. A method according to claim 18, wherein the ibuprofen and paracetamol is administered or ingested in the same way at approximately 6 hourly intervals.

25. Use of paracetamol and ibuprofen in the preparation of a medicament for treating moderate or severe osteoarthritis or moderate or severe rheumatoid arthritis, wherein the medicament comprises a combination composition having a synergistic therapeutically effective amount of ibuprofen and paracetamol in a ratio of approximately 125-150:approximately 475-500 respectively.

26. A method of treating moderate or severe osteoarthritis or moderate or severe rheumatoid arthritis comprising administering, or receiving, a synergistic therapeutically effective amount of paracetamol and ibuprofen substantially simultaneously in a ratio of approximately 475-500:approximately 125-150 respectively.
